(19) United States
(12) Patent Application Publication
(10) Pub. No.: US 2025/0160905 A1
(43) Pub. Date: May 22, 2025

(54) Scoliosis Correction Device Automatically and Synchronously Adjusted along with Spine Growth

(71) Applicants: Cheng Ching Yeh Wang, Taipei City (TW); Paonan Biotech Co., Ltd., Taipei City (TW)

(72) Inventor: Chung Chun Yeh, Taipei City (TW)

(21) Appl. No.: 18/656,738

(22) Filed: May 7, 2024

(30) Foreign Application Priority Data
Nov. 22, 2023 (TW) 112145082

(51) Int. Cl. A61B 17/70

(52) U.S. Cl. CPC A61B 17/7046; A61B 17/7005

(57) ABSTRACT

A scoliosis correction device has a plurality of saddles each having a guiding hole formed through the saddle, and secured to a pedicle; and at least a correction rod engageable within the guiding hole in the saddle; whereby upon spinal growth of a patient, the saddle or saddles as secured to the pedicle or pedicles will be moved in the correction rod, so that the correction device will be automatically and synchronously adjusted for keeping pace with the spinal growth of the patient. This correction device includes a saddle integrally formed for reducing the elements in construction of the correction device for saving cost and shortening surgery time for reducing operation risk.

### BACKGROUND OF THE INVENTION

In a traditional growing rod (TGR), the growing rod consists of proximal and distal anchors of pedicle screws attached to the spine, and two vertical rods attached to the anchors. The distal and proximal rods are connected by a telescopic unit for distraction or lengthening for correcting the deformed spine. The lengthening procedure is performed every six months, involving a surgical incision in the patient's back. Such repeated surgical lengthenings significantly increase the risk of complications. Also, it may cause pain either physically or psychologically to the patient.

So, a magnetically-controlled growing rod (MCGR) is achieved without the need for invasive surgical lengthening. Magnetically-controlled growing rods are adjustable rods placed next to the spine during surgery. The rods expand by using magnets and an external remote controller. Even the MCGR may overcome the drawbacks of repeated surgical lengthenings as required by TGR, the patient, due to his or her continuing growth, the spine still requires frequent adjustments, along with the patient's growth. Such adjustments of MCGR must be manipulated by a professional doctor, without being automatically adjusted, thereby still causing inconvenience for the patient.

U.S. Pat. No. 11,744,618 B2 disclosed a spinal correction tensioning system, which includes a flexible cord, configured for tensioning between two vertebral anchors for applying correctional tension force to the spinal column. However, it still requires a spring member (35) for adjusting the compression of the spring member (35), indicating that surgical incisions for adjusting the spring member (35) are still required to thereby cause complication or pain to the client.

The present inventor has overcome the defects as found in the prior arts, and invented this scoliosis correction device.

### SUMMARY OF THE INVENTION

The object of the present invention is to provide a scoliosis correction device comprising: a plurality of saddles each having a guiding hole formed through the saddle, and secured to a pedicle; and at least a correction rod slidably engageable within said guiding hole in each said saddle; whereby upon spinal growth of a patient, the saddle or saddles as secured to the pedical or pedicles will be moved in said correction rod, so that the correction device will be automatically and synchronously adjusted for keeping pace with the spinal growth of the patient.

Another object of the present invention to provide a correction device includes a saddle integrally formed for reducing the elements in construction of the correction device for saving cost. Therefore, the correction device can be assembled easily and quickly so as to shorten the surgery time to reduce operation risk.

Other objects of the present invention may be referred to the detailed description as hereinafter described.

### BRIEF DESCRIPTION OF THE DRAWING

FIG. 1 shows an exploded view of a first preferred embodiment of the present invention.

FIG. 2 is an assembled illustration of FIG. 1.

FIG. 3 is a plan view of the first embodiment when installed on a patient's spine.

FIG. 4 is an illustration when securing the correction rod on a patient's pedicle.

FIG. 5 is an illustration showing the bending movement of the present invention.

FIG. 6 is an exploded view of a second preferred embodiment of the present invention.

FIG. 7 shows an assembled device of FIG. 6.

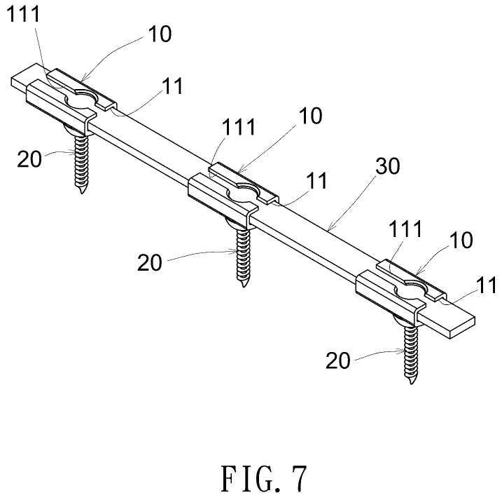

FIG. 8 is a plan view when assembling the present invention on a spine.

FIG. 9 shows a third preferred embodiment of the present invention.

FIG. 10 is an assembled illustration from FIG. 9.

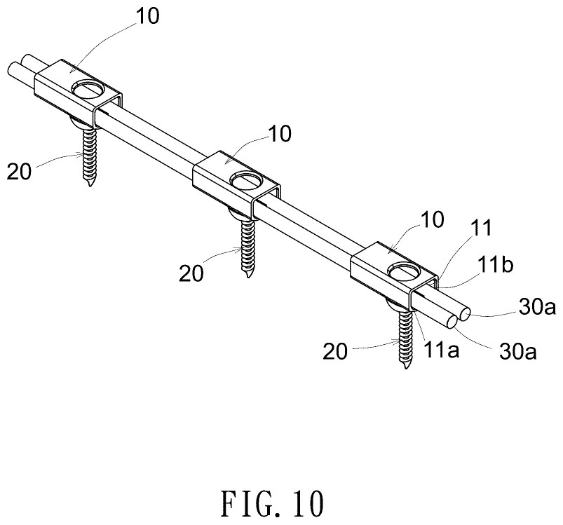

FIG. 11 is a plan view when assembled on a spine.

FIG. 12 is a perspective view of a fourth preferred embodiment.

FIG. 13 shows an assembled device from FIG. 12.

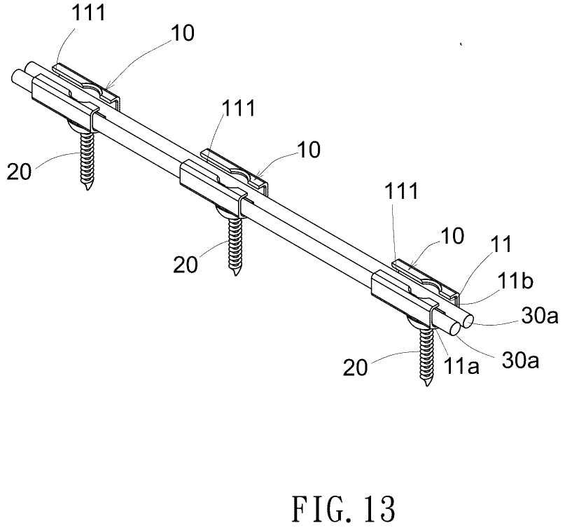

FIG. 14 shows a fifth preferred embodiment.

FIG. 15 shows an assembled device from FIG. 14.

FIG. 16 shows a sixth preferred embodiment.

FIG. 17 is a plan view when the device of FIG. 16 is assembled on a patient's spine.

FIG. 18 shows a seventh preferred embodiment.

FIG. 19 shows an assembled device from FIG. 18.

FIG. 20 shows a guiding saddle in accordance with the present invention.

FIG. 21 shows a coupling saddle of the present invention when connected to a spine fusion device.

### DETAILED DESCRIPTION

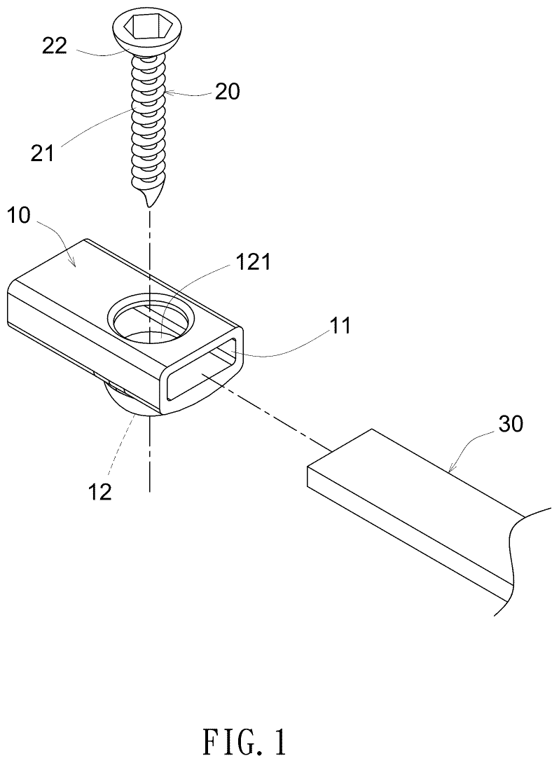

As shown in FIGS. 1˜5, a scoliosis correction device as automatically and synchronously adjusted along with spine growth comprises: a plurality of saddles 10; and pedicle screws 20 each adapted to be fixed on a pedicle P; a correction rod 30 fixed on one pedicle P of the spine vertebra for slidably adjusting the saddle 10 on the correction rod 30 to synchronously keep a pace with the spinal growth of a patient. Each saddle 10 is longitudinally formed with a guiding hole 11 through the saddle 10, and is latitudinally formed with a connecting hole 12 which is passing through the saddle 10 to be perpendicular to the guiding hole 11. A pedicle screw 20 is provided to pass through the connecting hole 12 to be fixed into the pedicle P, allowing a movable adjustment of the saddle 20 on the correction rod 30 along with the patient's spinal growth, thereby preventing frequent surgeries for the spinal correction. The saddle 10 may be integrally formed.

Since the scoliosis correction of the present invention can be automatically and synchronously adjusted to keep pace with the patient's spinal growth to overcome the defects of the prior arts by simplifying the elements in construction of the correction device, to reduce the installation procedures and to shorten the surgery time to reduce the patient's pain, thereby rendering the novelty and unobviousness of the present invention.

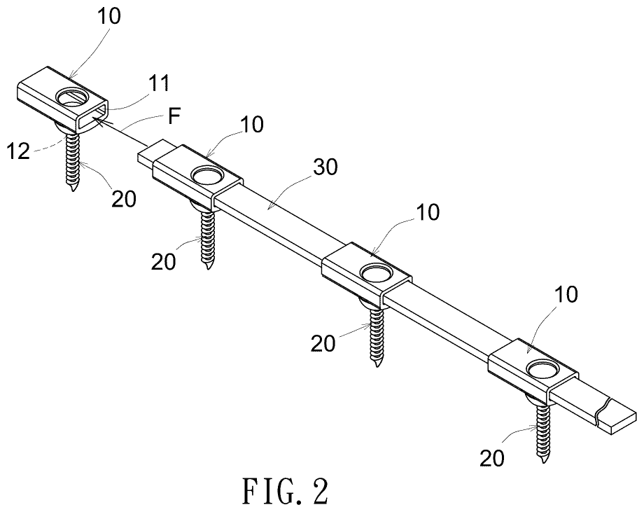

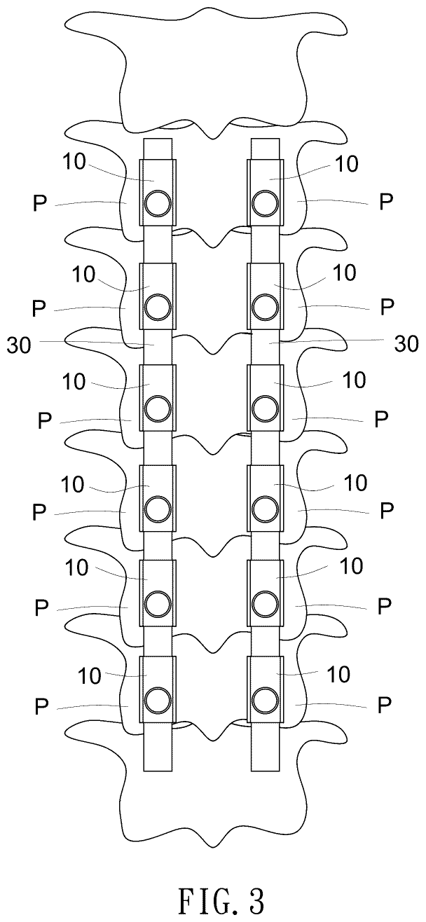

As shown in FIGS. 2, 3 and 5, the correction rod 30 and guiding hole 11 each has a cross section of a flat rectangular shape so as to allow each saddle 10 to be adjustably moved along the correction rod 30 to allow a patient's frontward or rearward bending or to allow his or her leftward or rightward twisting at a permitted angle. However, such a correction rod 30 formed as flat rectangular shape will not allow a sideward bending of the patient's spine so as to prevent from deformation or dislocation of the spinal vertebra after being corrected by the present invention. So, the present invention allows a proper movement of the patient for his or her life quality, merely causing a little inconvenience for the patient.

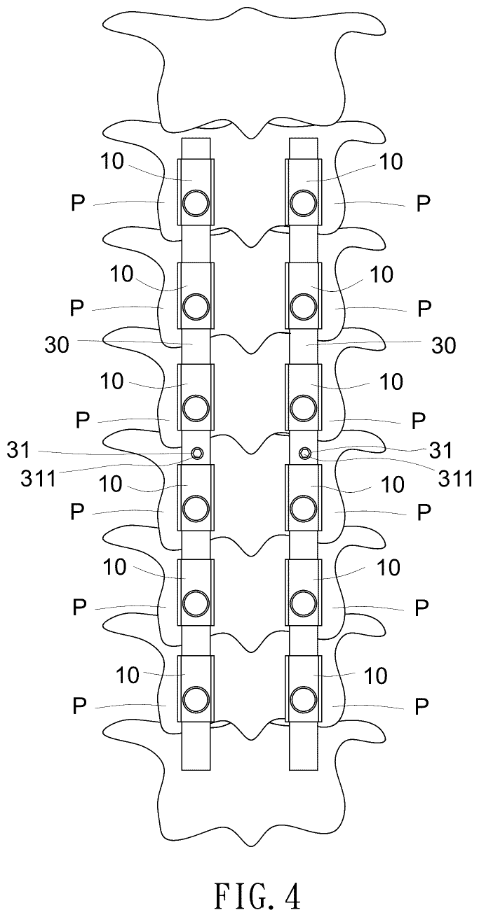

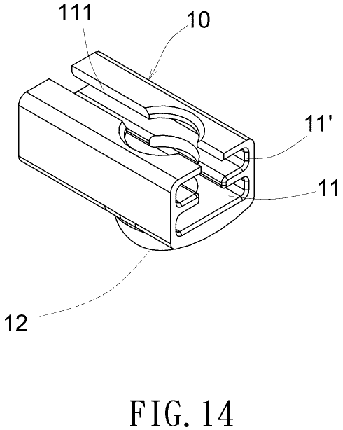

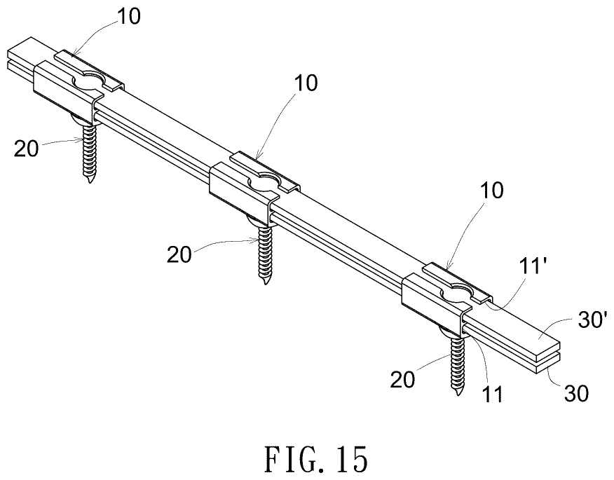

As shown in FIG. 4, the correction rod 30 is formed with a screw hole 31 so that a fixing screw 311 may be provided for fixing the correction rod 30 on a specific pedicle P to prevent from escape of the rod 30 from the saddle 10. As shown in FIGS. 14 and 15, each saddle 10 is formed with a guiding hole 11 through the saddle 10 and further formed a juxtaposed guiding hole 11′ formed through the saddle 10 as parallel and juxtapositional to the guiding hole 11 to respectively insert the correction rod 30 and a juxtaposed correction rod 30′ juxtapositional to the correction rod 30 through the guiding hole 11 and the juxtaposed guiding hole 11′ to prevent the correction device from being bent sidewardly, and to maintain the spine as corrected at a precise corrected position.

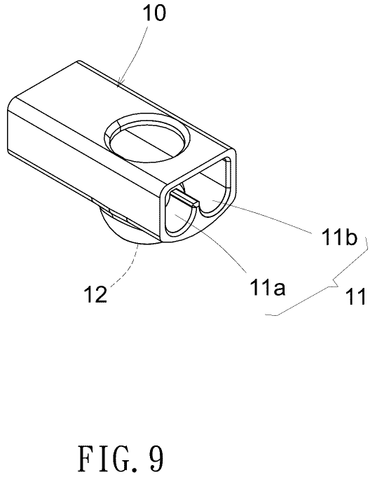

As shown in FIGS. 9˜13, each guiding hole 11 includes a pair of arcuate grooves 11a, 11b to respectively insert two correction rods 30a into the pair of arcuate grooves 11a, 11b. Each groove 11a or 11b has an opening smaller than a diameter of the rod 30a so as to stably engage the rod within the saddle 10. In practical application, one or two correction rods 30a may be provided in the present invention. If two rods 30a are provided, one rod 30a may have its material, elasticity, hardness to be different from another rod 30a for optional choice by the patient.

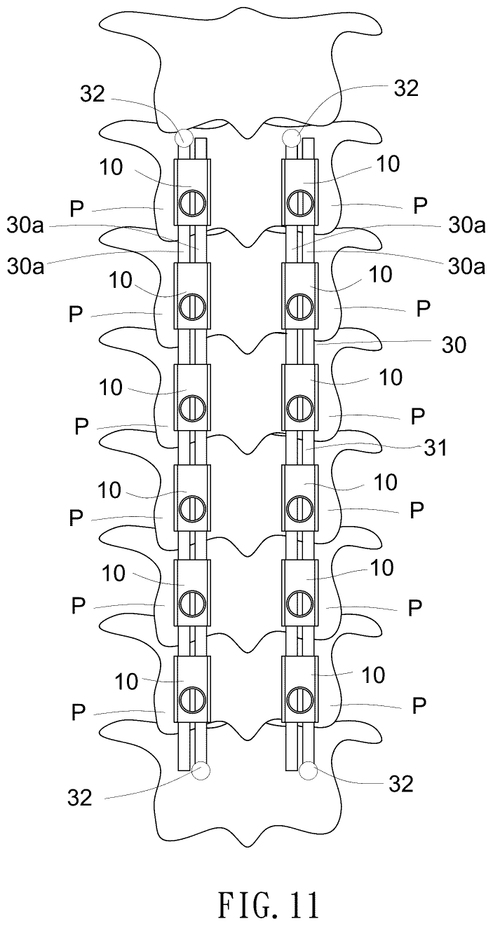

As shown in FIG. 11, a screw is provided to fix the upper end 32 of an upper pedicle P, while another screw is provided to fix the lower end 32 of the rod 30a on a lower pedicle P.

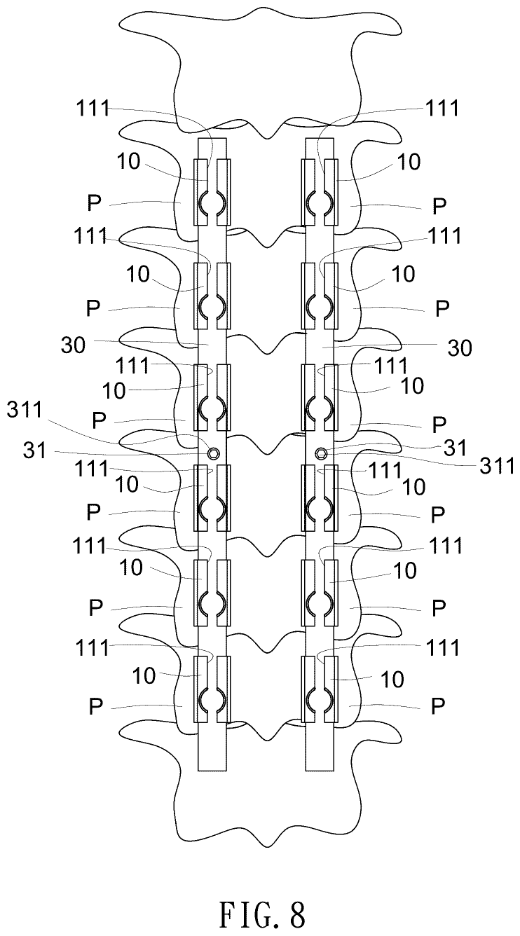

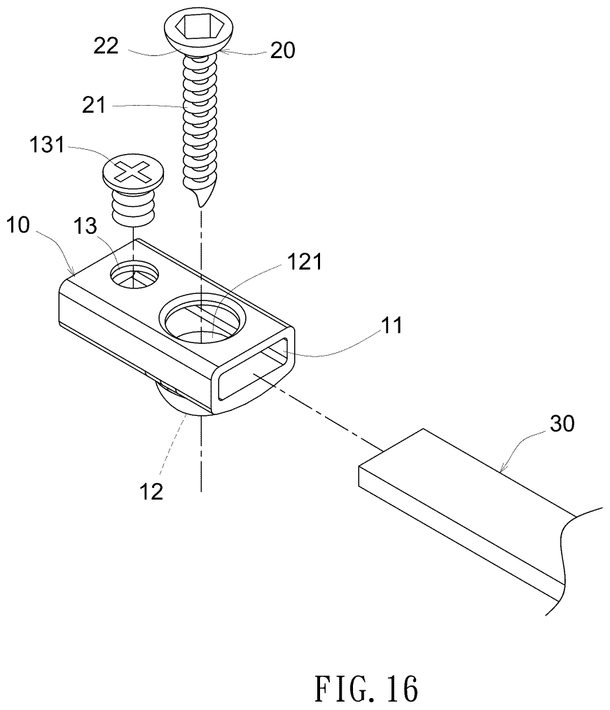

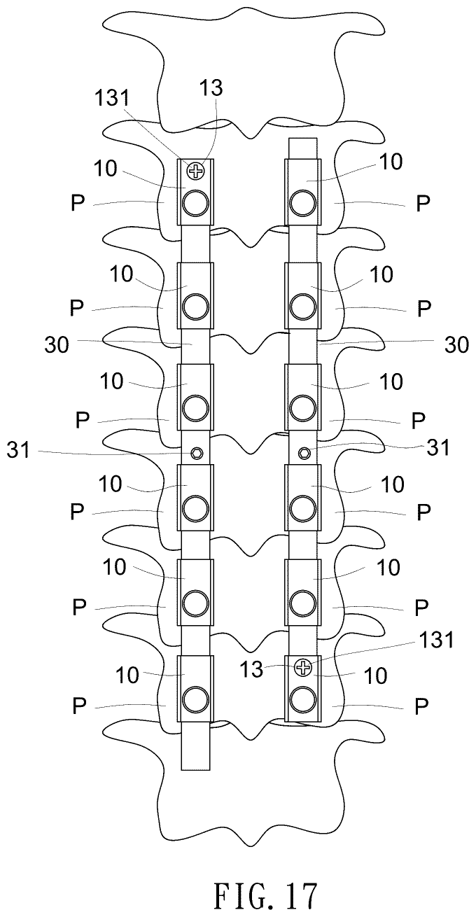

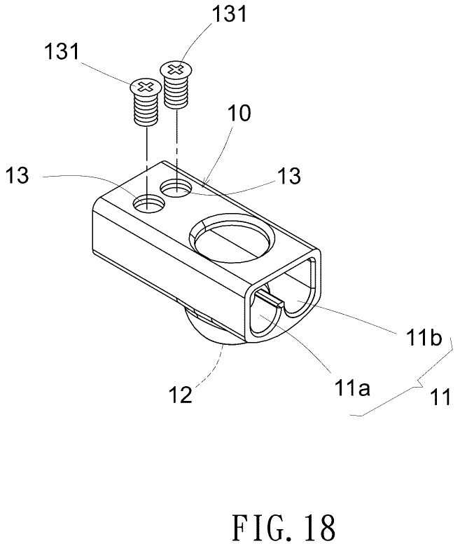

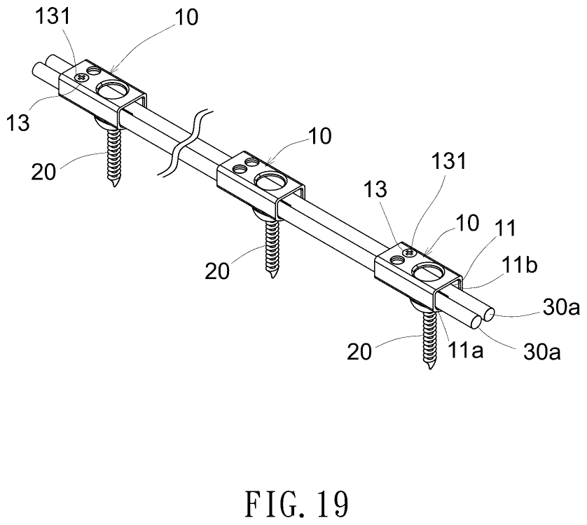

As shown in FIGS. 16˜19, a saddle 10 may be formed with a fixing hole 13 therethrough so that a fixing screw 131 is provided through the fixing hole 13 to fix the correction rod 30 or 30a with the saddle 10 to prevent from unexpected sliding movement of the correction rod 30 or 30a. As shown in FIG. 17, one correction rod 30 may have its upper end fixed with an upper saddle 10 as shown on the upper left side of the spine of FIG. 17, and another correction rod 30 may have its lower end fixed with a lower saddle 10 as shown on a lower right side of the spine. This arrangement may allow each correction rod 30 to be moved as synchronously keeping pace with the spinal growth, and may allow each saddle 10 to be stably slid along each correction rod during the spinal growth. As shown in FIGS. 18 and 19, when a pair of correction rods 30a are provided, one correction rod 30 may have its upper end secured to a saddle 10 positioned on an upper portion of the spine, and another correction rod 30 may have its lower end secured with a saddle positioned on a lower portion of the spine. The correction rod 30 or 30a may be secured to a pedicle at a specific position of spine, such as in middle portion of spine shown in FIGS. 4, 8, and 17; or secured to a pedicle at either an upper end or a lower end of spine as shown in FIG. 11, depending upon the practical requirement of the patient.

The correction rod 30 may be secured to a top vertebra of the spire or a bottom vertebra. For example, after fixing the saddle 10 on a pedicle as shown in FIG. 2, the correction rod 30 may then be inserted through each guiding hole 11 of each saddle 10 (direction F). The patient's incision on his or back should be as minimal as possible to minimize the incision area to help sooner healing, minor complication and reduce the hospitalization time.

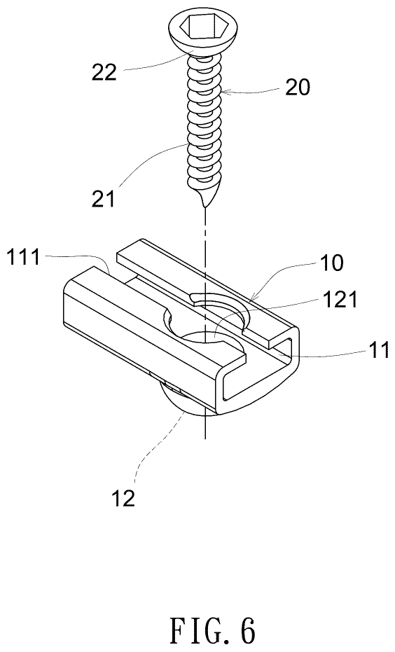

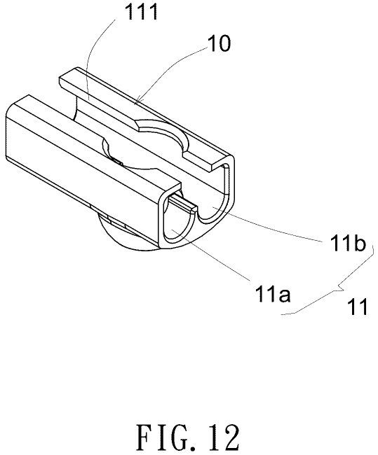

As shown in FIGS. 6˜8 or FIGS. 12˜15, each saddle 10 may be formed with a slit 111 through the saddle to be communicated with each guiding hole 11. This may help a quicker insertion of the correction rod 30 into the saddles 10; the above structure is suitable for patients who require a complete opening of the spinal area for surgical correction. Please be noted that the rod 30 may have a proper flexibility so that the rod 30 is tilted from above the slit 111 and may be easily and smoothly inserted into the guiding hole 11 to be engaged into the saddle 10.

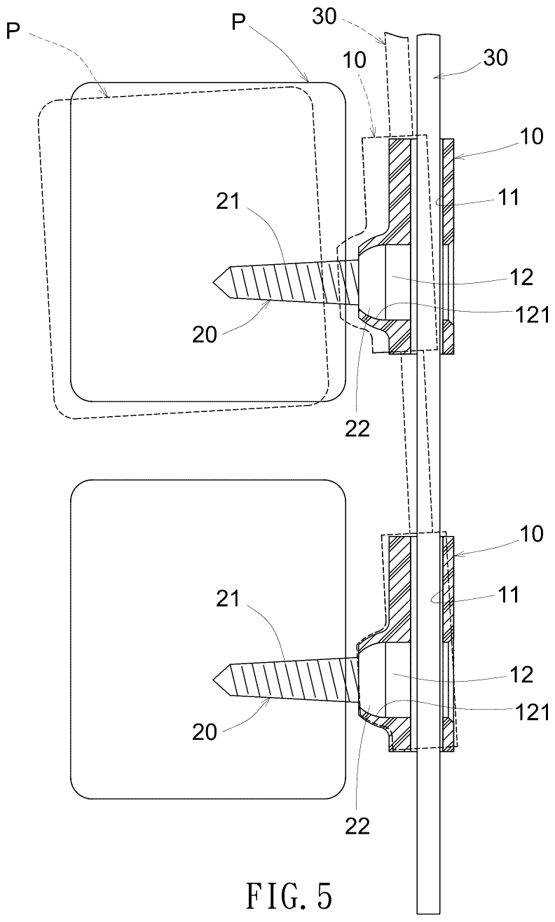

As shown in FIG. 5, the connecting hole 12 has its bottom portion formed with a spherical cavity 121 to be engaged with a spherical bottom portion of a head portion 22 of the pedicle screw 20, of which a shank portion 21 of the pedicle screw 20 is fixed into pedicle P of vertebra, thereby allowing a bending or twisting movement of the patient.

When installing the present invention, the spinal vertebra may be corrected to a right position and then the correction device may be installed on both sides or a single side of the patient's spine, depending upon his or her requirement.

The correction rod 30 or 30a may be made from: titanium alloy or other memory alloy; or made from non-metallic materials, such as Polyether Ethere Ketone (PEEK) composite, but not limited.

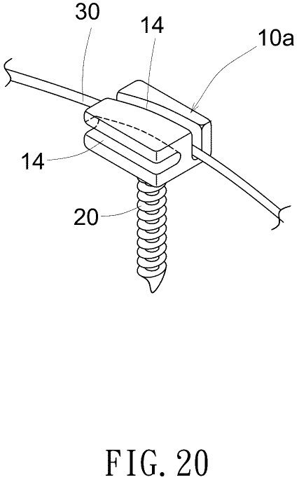

As shown in FIG. 20, the saddle is modified to be a guiding saddle 10a having at least a guiding groove 14 formed on the saddle 10a to be slidably engaged with the correction rod 30 in the groove 14a after the saddle 10a is secured on a patient's pedicle. The guiding groove 14 may be an arcuate groove or a tilting groove, adapted for acruately engaging a correction rod.

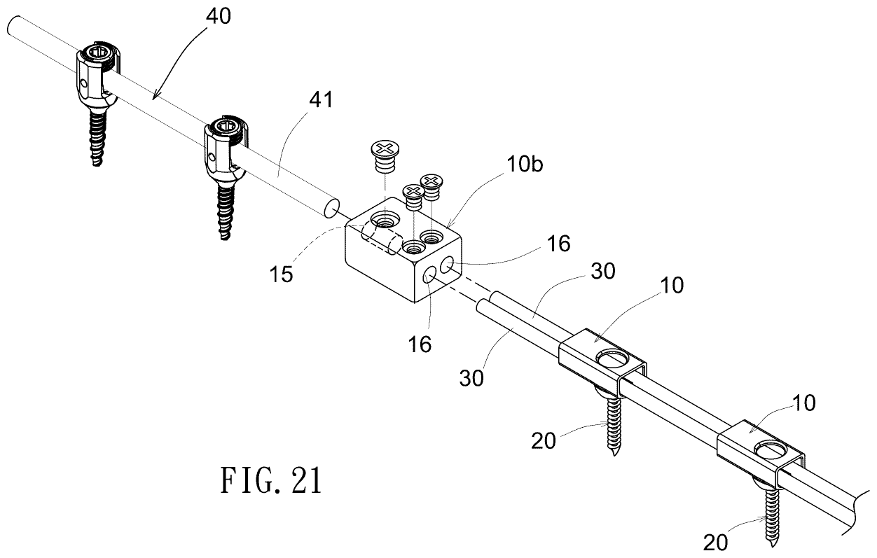

As shown in FIG. 21, the saddle is modified to be a coupling saddle 10b which is secured to a pedicle, having a first connector 15 and at least a second connector 16 for connecting at least one correction rod 30 of the present invention. Therefore, a partial spine may be provided with the spine fusion device 40, while the remaining spine may be implemented with the correction device of this invention for simultaneously performing a spine fusion, and also for providing the scoliosis correction device of this application to be automatically adjusted along with the spinal growth of the patient.

I claim:

1. A scoliosis correction device comprising:
a plurality of saddles each having a guiding hole formed through the saddle, and secured to a pedicle; and at least a correction rod slidably engageable within said guiding hole in each said saddle; whereby upon spinal growth of a patient, the saddle or saddles as secured to the pedicle or pedicles will be moved in said correction rod, so that the correction device will be automatically and synchronously adjusted for keeping pace with the spinal growth of the patient.

2. A scoliosis correction device according to claim 1, wherein said correction rod has a cross section formed as a flat rectangular shape, and said guiding hole of said saddle has its cross section formed as a flat rectangular shape.

3. A scoliosis correction device according to claim 1, wherein said correction rod is formed with a screw hole therein adapted for passing a pedicle screw therethrough for securing the rod on the pedicle.

4. A scoliosis correction device according to claim 1, wherein said saddle is formed with a first guiding hole for inserting a first correction rod therein and a second guiding hole parallel to and juxtaposed to said first guiding hole, said second guiding hole adapted for inserting a second correction rod therein.

5. A scoliosis correction device according to claim 1, wherein said guiding hole includes two grooves longitudinally recessed in said guiding hole, each said groove has a section of arcuate shape.

6. A scoliosis correction device according to claim 1, wherein said saddle is longitudinally formed with a slit which is communicated with said guiding hole.

7. A scoliosis correction device according to claim 1, wherein said saddle is formed with a connecting hole through a bottom portion of said saddle adapted for fixing a pedicle screw, through said connecting hole, into a pedicle; and said pedicle screw includes a head portion and a shank portion having a spherical bottom portion of the head portion to be engageable with a spherical cavity formed in a bottom portion of said connecting hole.

8. A scoliosis correction device according to claim 1, wherein said saddle is formed with a fixing hole for passing a fixing screws for securing said saddle on said correction rod.

9. A scoliosis correction device according to claim 1, wherein said saddle is a guiding saddle having a guiding groove formed therein for guiding the correction rod in said guiding groove.

10. A scoliosis correction device according to claim 1, wherein said saddle is a coupling saddle, having a first connector for connecting a spinal fusion device; and a second connector opposite to said first connector for connecting at least a correction rod with said second connector.
